(19) United States
(12) Patent Application Publication
(10) Pub. No.: US 2005/0246032 A1
(43) Pub. Date: Nov. 3, 2005

(54) Bone and tissue implants and method of making

(75) Inventors: Jack C. Bokros, Austin, TX (US); Robert B. More, Austin, TX (US); James A. Accuntius, Georgetown, TX (US)

Correspondence Address: James J. Schumann;Fitch, Even, Tabin & Flannery, Suite 1600, 120 South LaSalle Street, Chicago, IL, 60603-3406, US

(73) Assignee: Medical Carbon Research Institute, Austin, TX (US)

(21) Appl. No.: 11/018,559

(22) Filed: Dec. 20, 2004

Related U.S. Application Data
(63) Continuation of application No. PCT/US03/19578, filed on Jun. 20, 2003.
(60) Provisional application No. 60/390,450, filed on Jun. 21, 2002.

(51) Int. Cl. A61F 2/28

(52) U.S. Cl. 623/23.6; 623/901; 427/2.26

(57) ABSTRACT

A bone implant (71) comprises a body formed of an articulated open cell structure of a lightweight material, the surfaces of which structure are covered with a thin metal layer. A layer of biocompatible pyrocarbon coating is applied to the metal-coated structure so as to cover the entire structure and provide a dense, nonporous, biocompatible layer. Pyrocarbon is then selectively removed from portions (73) of the surface of the body to expose sections of the original surface which lead to regions of interconnected channels into which bone and tissue ingrowth are promoted while end regions (75) and (77) remain totally covered with such pyrocarbon.

This application is a continuation of International Application Serial No. PCT/US2003/019578, filed Jun. 20, 2003, and claims priority from Provisional Application Ser. No. 60/390,450, filed Jun. 21, 2002, the disclosures of both of which are incorporated herein by reference.

### FIELD OF THE INVENTION

This invention relates to bone and other prosthetic implants, and more particularly to bone implants that are designed for implantation into cancellous or trabecular bone and to other prosthetic implants into which tissue ingrowth is desired, as well as to methods of making such implants.

### BACKGROUND OF THE INVENTION

A need has long existed for better porous biomaterials that are structurally strong and that can be used as implants in reconstructive orthopedics and for other tissue applications. Porous polymeric materials and porous ceramics which have previously been tried are not believed to generally incorporate adequate mechanical properties. More recently, a highly porous, tantalum surface biomaterial having excellent physical, mechanical and tissue ingrowth properties has been developed; such is generally described in U.S. Pat. No. 5,282,861, issued Feb. 1, 1994, entitled “Open Cell Tantalum Structures for Cancellous Bone Implants and Cell and Tissue Receptors.” It is felt that the structure of this material mimics the microstructure of natural cancellous or trabecular bone materials. Trabecular bone is a generally spongy substance that has a reticulated structure, which is recreated in these open cell tantalum structures. This new structure is manufactured by creating a thin foam substrate of carbonaceous material and then, through CVD and/or CVI, depositing tantalum metal on all of the surfaces so as to create a substantially tantalum structure having only an underlying, thin, totally enveloped framework of the original carbon substrate.

In an article written by J. Dennis Bobyn, Ph.D. in Orthopedics, 22, 9 pp. 810-812 (September 1999) entitled “The Good, Bad and Ugly: Fixation in Bearing Surfaces for the Next Millennium”, a variety of implants having fixation and bearing surfaces were reviewed. This new porous tantalum biomaterial was felt to be a good example of using improved technology to create materials useful in orthopedic surgical reconstruction procedures. The product is described as one made by chemical vapor infiltration of tantalum onto a glassy or vitreous carbon substrate that creates a tantalum microtexture on the myriad of struts that form the material, resulting in an ultimate topography that implant study has shown to be osteoconductive. It was reported that such commercially available porous tantalum structures had an overall porosity of 75% to 80% and that such allowed a greater volume of bone ingrowth and faster development of fixation strength in an implant. For bearing surfaces, compression-molded polyethylene was suggested, as the porous tantalum structure itself is not suitable for a surface where articulation will occur. Although high density polyethylenes have gradually improved, such polymeric materials have an inherent tendency to spawn fine particles as a result of abrasion; thus, although implantation characteristics may be excellent, the bearing surface remains less than ideal.

Accordingly, improvements to provide more acceptable implants have continued to be sought, particularly those for bones having bearing surfaces, which are made of porous metal biomaterials that will promote the ingrowth of trabecular bone.

### SUMMARY OF THE INVENTION

It has now been found that bone and tissue implants can be effectively created using a porous metal biomaterial, such as a metal-covered reticulated substrate, e.g. the commercially available tantalum porous biomaterials, and applying a pyrocarbon coating of suitable characteristics that will totally seal the exterior surface of the porous biomaterial and render it totally compatible with hard bone tissue and body fluids. Thereafter, in the regions of the implant that will interface with trabecular bone and selected tissue, the deposited carbon is selectively removed in a manner that does not adversely affect the underlying metal, as by using electrodischarge machining (EDM), thereby reopening these regions of highly porous reticulated structure for future bony ingrowth thereinto. The thickness of the pyrocarbon coating, which is preferably strong, hard and tough, that is applied is such that the coated regions of the implant are adequate to excellently serve as an articulating or bearing surface, and such surfaces are preferably highly polished to produce a hard surface essentially free of surface irregularities that interfaces excellently with natural bone or other biomaterials as a part of a joint.

By using this manufacturing procedure, the invention provides a bone implant designed for implantation into a region where there will be an interface with trabecular bone, wherein a selective region will have (as the result of the removal of the pyrocarbon coating that previously covered it) a highly porous metal, e.g. tantalum, structure that is highly conducive to bony ingrowth, while the remainder of the implant is substantially nonporous, being covered with a continuous layer of hard, biocompatible pyrocarbon that has properties that render it compatible with hard bone tissue and body fluids. When the implant is to be part of an articulating joint, a portion of the nonporous surface can be polished to create a smooth, hard, tough surface region having low surface irregularities.

In one particular aspect, the invention provides a bone or tissue implant which comprises a body formed of a reticulated open cell substrate of lightweight material having open spaces in the form of a network of interconnected channels, a thin film of metal covering the surfaces of said lightweight material throughout the network of interconnected channels, and a layer of biocompatible pyrocarbon coating a large portion of the exterior surface of said body so as to render such exterior surface bone and tissue-compatible, wherein there is a region of the exterior surface from which said pyrocarbon layer has been removed to expose said metal-covered reticulated substrate and thereby promote bony and/or tissue ingrowth into such exposed region.

In another particular aspect, the invention provides a method for making a bone or tissue implant designed for implantation in the human body, which method comprises the steps of coating a body having the desired shape for such implant over its entire exterior surface with pyrocarbon that is biocompatible, which body is formed of a reticulated open cell structure of a lightweight metallic biomaterial having open spaces in the form of a network of interconnected channels, said coating being carried out under conditions to provide a coating over substantially the entire exterior surface of said body in a manner so that the resultant pyrocarbon has characteristics that render it bone and tissue-compatible, and selectively removing said pyrocarbon coating from regions of said body to expose said open cell reticulated structure and thereby promote bony and/or tissue ingrowth into such selected regions when said body is implanted in association therewith.

### BRIEF DESCRIPTION OF THE DRAWINGS

FIG. 1 is cross-sectional view through a percutaneous implant designed for administration of pharmaceuticals to a living body; it has a stem portion which interconnects a head designed to reside at skin level and a stabilizing lower flange.

FIG. 2 is a cross-sectional view of an orthopedic prosthesis embodying various features of the invention having a head having a portion designed to function as an articulating surface and a stem for implantation into trabecular bone.

FIG. 3 is a perspective view showing an intervertebral disk prosthesis having a pair of flanking end sections coated with biocompatible pyrocarbon and having a center section designed to facilitate bony ingrowth, which disk embodies various features of the invention.

FIG. 4 is a perspective view showing a valve body for a prosthetic heart valve having exterior and interior surfaces coated with biocompatible pyrocarbon except for an exterior surrounding fastening ring that is designed to facilitate tissue ingrowth.

### DETAILED DESCRIPTION OF THE PREFERRED EMBODIMENTS

Pyrolytic carbon having certain characteristics was found to be highly biocompatible several decades ago and since then has been used in the construction of a large number of prosthetic heart valves and other medical devices where compatibility with blood is of primary importance to avoid clotting and the like. The early pyrocarbons that were employed, to obtain the desired physical and chemical characteristics, included silicon as an alloying agent; however, it was discovered in the early 1990's that, by using very selective coating conditions, pure unalloyed pyrocarbon could be produced having improved mechanical properties, such as strength and toughness. Processes for making such unalloyed, isotropic biocompatible pyrocarbon are disclosed and claimed in U.S. Pat. No. 5,514,410, and this carbon is commercially available today as On-X® carbon.

It has now been found that it is feasible to coat substrates made of a reticulated open cell structure of lightweight metal-covered material with biocompatible pyrocarbon, and more specifically with unalloyed pyrocarbon having the mechanical characteristics of On-X® carbon; this is feasible even when the open spaces constitute a major portion of the volume of the substrate and are in the form of a network of interconnected channels that extend throughout the network. More particularly, it has been found that not only can such structures, such as vitreous carbon, covered entirely with a thin film or layer of metal, such as tantalum, be subjected to high temperature coating processes to deposit pyrocarbon without difficulty, but that the pyrocarbon deposited will effectively completely seal the highly porous surface of such a substrate and can be accumulated to a surface thickness such that it can be polished to provide a hard, strong, tough surface having few surface irregularities that is well-suited to constitute an excellent articulating surface. Even more importantly, this coating procedure can be accomplished without having the pyrocarbon penetrate too deeply, e.g. not more than about 1 millimeter, so that it can be selectively removed and returned to its original character in regions where the bone or tissue ingrowth will be desired.

Another desirable characteristic of the preferred substrate material, in addition to its being excellently suitable for bony ingrowth, is that it has an overall modulus very close to that of human bone; thus, when the resultant product is used as a bone implant, it is totally compatible from the standpoint of its mechanical characteristics, such as the distribution of stresses. Suitable open cell structures, based upon vitreous or glassy carbon foam starting materials, are commercially available under the trademark Hedrocel by the Implex Corporation of New Jersey, U.S.A., and these are preferred. Such an original vitreous carbon framework is coated with a suitable metal film, preferably tantalum or niobium; however, other equivalent metals, e.g. titanium, might alternatively be used, as well as alloys of any of these. Other suitable metal biomaterials may alternatively be used which have moduli and biocompatibility similar to that of such tantalum-coated structures, for example aluminum oxides, so long as they can withstand high temperature pyrocarbon coating.

Because the depth of pyrocarbon penetration that occurs during the deposition process that is used is limited, it can be thereafter selectively removed in regions where bony ingrowth or tissue ingrowth is desired by processes that can effect such efficient removal without adverse effect upon the underlying metal film. Generally, bone implants made in this fashion find particular use in the area of small joints where trabecular bone growth into a stem portion is desirable; however, there are expected to likewise be opportunities for employment of these implants in the area of femoral heads, radial heads and the like. Other areas of interest are those where the implants would be inserted at cartilage wear points. Spinal inserts, such as intervertebral disks, is another region of interest, and various rod-like shapes that would be inserted into bones for connective purposes are likewise expected to have particular interest.

From the standpoint of areas where tissue ingrowth is desired, one area is that of heart valve bodies where a flexible sewing cuff is presently employed for securing the heart valve to the surrounding tissue, generally with sutures applied by the surgeon; it is felt that promotion of the tissue ingrowth into such a substitute sewing cuff made of a reticulated design would be a very desirable alternative. Venous tubes of such a structure coated with pyrocarbon can be designed to be secured in a specific tissue region for internal blood flow; all or part of the exterior surface would have pyrocarbon selectively removed therefrom to create tissue ingrowth conducive surfaces that would stabilize the object over time.

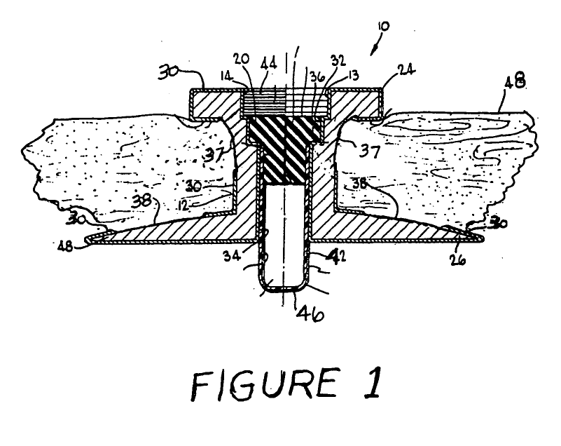

Illustrated in FIG. 1 is a percutaneous implant device which is adapted for controlled and continuous percutaneous administration of medication to a living body. The implant device 10 includes a stem 12 having a passageway 14 therethrough, a large stabilizing flange 26 at the base of the stem and a valve 20 within the stem passageway. An upper head or flange 24 surmounts the stem. The passageway is of greater diameter at its upper end 13, which is located above an intermediate zone 32 of slightly smaller diameter and a lower zone 34 that extends down through the stabilizing flange. Female threads 44 provided at the interior surface of the upper portion of the passageway facilitate interconnection with a medication-injecting device. The stem 12, the upper flange 24 and the subcutaneous stabilizing flange are all formed as a single unit from a porous biomaterial substrate, preferably one in the form of a metal-covered reticulated material, such as the commercially available, tantalum, porous biomaterial which is sold under the trademark Hedrocel. After machining to the desired configuration, the Hedrocel substrate is coated entirely with pyrolytic carbon 30, preferably On-X® carbon, and then pyrocarbon is selectively removed from two regions to facilitate the desired ingrowth of tissue. The first region 37 extends from the undersurface of the upper flange 24 to a location near the midpoint of the stem. The second region 38 is an annular region on the upper surface of the stabilizing flange 26. The passageway through the lower zone of the implant is designed to support a porous generally tubular dispenser 42 which is constructed to provide controlled and continuous medicinal release at a predetermined rate through its sidewall. The upper end of the tube-shaped dispenser is open and is affixed to the interior of the passageway and thus creates a reservoir for drugs or other medication at the closed bottom 46 of the dispenser. Medication is supplied through the valve, and once in the reservoir, it is released through the porous sidewall into the surrounding subcutaneous tissue 48 of the living body in the desired controlled manner. The valve 20 may be a simple elastomeric plug which seals the upper end of the passageway; it may be made of silicone rubber or the like and fashioned to have a generally closed passageway that permits insertion of a hypodermic needle or the like therethrough.

Pyrocarbon is applied to the tantalum reticulated substrate sufficient to totally initially cover and seal all the exterior surfaces including the entire passageway region of the implant 10. The pyrocarbon deposit should have the characteristics described hereinafter, as such is highly biocompatible as has been proven over several decades. Following the coating operation, the substrate will be completely encased in pyrocarbon. Once coating is complete, pyrocarbon is removed from the two regions 37 and 38 so as to expose the tantalum reticulated substrate in these two areas and thus promote anchoring tissue growth, which has been shown to occur into the tantalum substrate material. Pyrocarbon removal is desirably carried out by EDM effectively reopens the regions of highly porous reticulated tantalum for the intended purpose of tissue ingrowth.

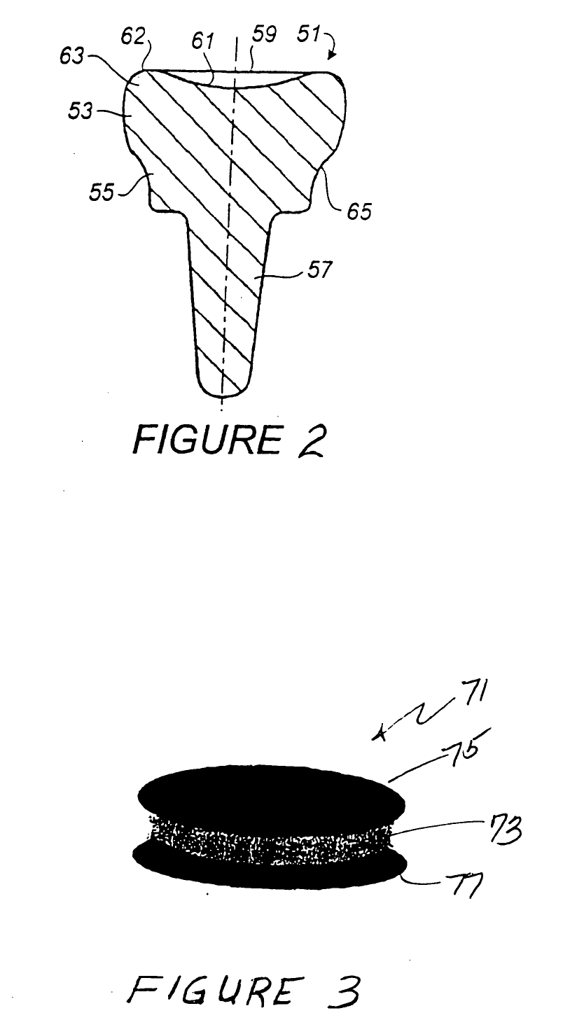

Shown in FIG. 2 is an insert 51 having a head 53 and an extended stem 57 that is designed to be implanted in trabecular bone to replace the end of the radius at the elbow. By using this construction, the task of cementing the stem 57 in place so that it will remain affixed over the life of the patient is greatly simplified because bony growth into the reticulated stem is promoted. The implant 51 includes a collar section 55 at the lower portion of the head 53 and just above the stem. The stem 57 is proportioned to be received within the medullary cavity of the radius, and accordingly after the coating of the entire implant prosthesis is completed, the pyrocarbon would be removed from the region of the stem 57, leaving the pyrocarbon coating on the entirety of the head. The head itself has a rim portion 62 and a peripheral surface 63 which advantageously remain covered with biocompatible pyrocarbon. The proximal surface 59 of the implant is formed with a shallow concave surface 61 which is designed for surface contact with the capitulum and is surrounded by the rim portion 62, with the shallow surface portion 61 constituting an articulating surface. Sufficient pyrocarbon would be deposited in this region so that polishing of the surface can be carried out to leave a polished dense hard, tough surface having surface irregularities not greater than 0.1 mm and preferably not greater than about 0.01 mm. This arrangement assures the long-lasting functioning of such a radial implant which becomes firmly secured to the bone through ingrowth at the stem because the polished pyrocarbon surface at the proximal end is tough and smooth and provides an excellent articulation surface. An implant of this general type might also be tailored to serve as a finger or toe implant at a joint, and accordingly such also would be formed with an appropriately shaped surface of the head that would be polished to provide a hard, strong, tough articulating surface.

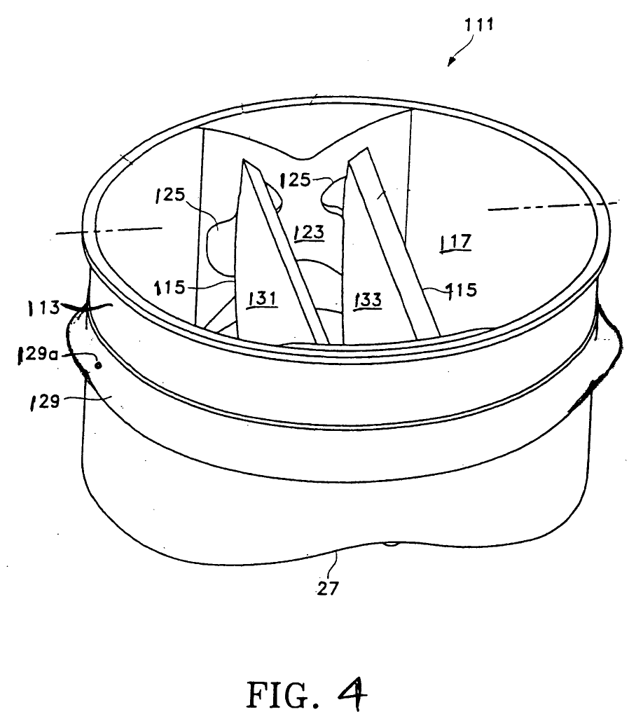

Illustrated in FIG. 3 is an intervertebral disk prosthesis 71 where a central section 73 of the disk is recessed and of a lesser diameter than the flanking end sections 75 and 77, which are of generally circular shape. The end sections 75, 77 would remain totally covered with biocompatible pyrocarbon following the coating operation so as to be biocompatible and nonporous. However, the central section 73, from which the pyrocarbon has been removed (as by EDM), comprises the open cell metal, i.e. tantalum, coated network of a highly porous configuration described hereinbefore that promotes tissue and/or bone ingrowth.

Illustrated in FIG. 4 is a ring-shaped housing 111 designed to serve as a heart valve body 113. Substantially, the entire surface of the body 113 is covered with biocompatible pyrocarbon so as to present an outer pyrocarbon surface similar as that described in U.S. Pat. No. 5,545,216. This annular valve body 111 carries a pair of pivoting leaflets 115 that prevent any substantial backflow of blood through the heart valve passageway by opening and closing as a result of pivoting in pairs of recesses 125 located in flat wall surface portions 123 of the otherwise cylindrical interior surface 117 of the annular body. The leaflets themselves have flat inflow and outflow surfaces 131 and 133. The exterior surface of the valve body 113 is provided with a bulbous, radially extending flange portion 129 that serves the purpose of the standard sewing ring for implanting the valve in the heart tissue from which the defective natural valve has been excised. The entire valve body may be made of a tantalum-reticulated material, such as that sold under the trademark Hedrocel, that is coated with pyrocarbon, preferably On-X carbon. This material is suitable to serve as a substrate, having sufficient resiliency it can be deformed so as to permit the insertion of the pair of leaflets in their operative locations. Alternatively, it may be feasible to construct a bulbous ring, with porous and nonporous surface sections, that would securely interfit about a specially constructed valve body.

After coating and polishing of the machined substrate takes place, the pyrocarbon is removed from the bulbous radial ring 129 to expose the reticulated tantalum material. If desired, passageways 129a can be provided in the bulbous ring to allow the passage of suture needles, or optionally a cloth suture ring can be located adjacent to the bulbous ring to facilitate the heart valve being sutured in place. In either instance, the reticulated metal, open cell structure provided by this integral surrounding bulbous ring facilitates secure placement of the valve body because tissue ingrowth in this area is positively promoted.

As an example of the preparation of one of these implants, a Hedrocel substrate is machined to serve as a radius implant 51 as shown in FIG. 2. The major portions of the head of the implant are machined so as to be undersized by about 0.015 inch, and it may be desired to slightly oversize the portions of the implant upon which pyrocarbon will be deposited and then removed so as to compensate for any slight decrease in size as a result of EDM. The substrate is coated for about two hours in a fluidized bed to apply about 0.015 inch of On-X carbon to the entire substrate using coating conditions as described in the '410 U.S. patent. The weight of the fluidizing bed is set to be about 10 times the total weight of the number of substrates being coated. A combined stream of fluidizing and hydrocarbon gas is maintained upward through the bed at about 25 standard liters per minute, with the stream being about 30 volume % propane and 70 volume % helium or argon, which will avoid any potential formation of tantalum nitride. Temperature and other coating conditions are carefully controlled to deposit pyrocarbon having a density between 1.7 and 2.1 grams per cm³, a diamond pyramid hardness of between about 200 and 250, a modulus of rupture for bending of at least about 58 psi×10³, and a K_{ic} of at least about 1.2 MPa (√ m). Once coating is completed, the substrates are submitted to automated mass finishing, as taught in U.S. Pat. No. 5,305,554. Thereafter, the pyrocarbon is removed from the areas of the stem 57 where it is desired that ingrowth of tissue should occur to anchor the implant in place. Pyrocarbon is preferably removed using conventional EDM; however, careful grinding techniques may alternatively be used to expose the underlying Hedrocel reticulated tantalum. After final machining, dimensional visual and quality inspections are performed, and the components are thereafter be cleaned and packaged for sterilization.

Although only a few illustrations of implants have been shown, it should be understood that this technology lends itself very well to a wide variety of orthopedic-type implants. Spinal disk replacements from the upper cervical to the lower lumbar are practical, and TMJ total joint replacement or partial joint replacement where the natural cartiledge will be in engagement with the carbon surfaces are excellent candidates. Total and partial shoulder replacements are feasible, including the humerus replacement in locations against the native glenoid, as well as localized repair of humerus or glenoid by using metal plug-like items. Total elbow replacement, as well as the radial head replacement described hereinbefore, is an excellent candidate for this technology, as is ulna head replacement. Partial or total finger joint replacements, such as the MCP and the PIP joints, are additional candidates. Femoral head replacement, localized repair of the femoral head, acetabulem repair, acetabular replacement, and partial or total knee replacements are other attractive candidates for this orthopedic material. Localized repair of the femur or the tibia, as well as total ankle replacement, are further candidates. Body nerve to artificial limb electrical connectors as well as porous metal biomaterial fixation items offer additional possibilities for use of these orthopedic devices. The field of dental implants, where the carbon surface will be present at the gingival line and the porous metal biomaterial surface will promote fixation into the bone, offer additional excellent possibilities for use of these implants.

Although the invention has been described with regard to certain preferred embodiments which illustrate the best mode presently known to the inventors for carrying out the invention, it should be understood that various changes and modifications as would be obvious to one having the ordinary skill in the art may be made without deviating from the invention which is defined in the claims appended hereto. The disclosure of the aforementioned U.S. patents and article are expressly incorporated herein by reference.

Particular features of the invention are emphasized in the claims that follow.

1. A bone or tissue implant which comprises:
a body formed of a reticulated open cell substrate of lightweight material having open spaces in the form of a network of interconnected channels,
a thin film of metal covering the surfaces of said lightweight material throughout the network of interconnected channels, and
a layer of biocompatible pyrocarbon coating a large portion of the exterior surface of said body so as to render such exterior surface bone- and tissue-compatible,
wherein there is a region of the exterior surface from which said pyrocarbon layer has been removed to expose said metal-covered reticulated substrate and thereby promote bony and/or tissue ingrowth into such exposed region.

2. The implant of claim 1 wherein said layer of pyrocarbon has a thickness sufficient to allow it to be polished and serve as an articulating surface in a bone joint.

4. The implant of claim 1 wherein said metal is tantalum.

5. The implant of claim 1 wherein said biocompatible pyrocarbon coating is pure unalloyed carbon having a density between 1.7 and 2.1 grams per cm³ and a diamond pyramid hardness of between about 200 and 250.

6. The implant of claim 1 wherein said biocompatible pyrocarbon has a modulus of rupture for bending of at least about 58 psi×10³ and a K_{ic} of at least about 1.2 MPa (√ m).

7. The implant of claim 1 wherein said body is an orthopedic prosthesis having a stem portion and a head portion wherein said stem portion is the region of the exterior surface from which said pyrocarbon layer has been removed.

8. The implant of claim 7 wherein said pyrocarbon layer has a thickness of at least about 0.2 mm and a region of the surface of said head portion is polished to provide an effective articulating surface for a bone joint.

9. The implant of claim 1 wherein said body is an intervertebral disk having a central portion and two end portions of greater diameter and wherein the surface of said central portion is said region from which said pyrocarbon layer is removed to expose said metal-covered open cell substrate.

10. A method for making a bone or tissue implant designed for implantation in the human body, which method comprises the steps of
coating a body having the desired shape for such implant over its entire exterior surface with pyrocarbon that is biocompatible, which body is formed of a reticulated open cell structure of a lightweight metallic biomaterial having open spaces in the form of a network of interconnected channels,
said coating being carried out under conditions to provide a coating over substantially the entire exterior surface of said body in a manner so that the resultant pyrocarbon has characteristics that render it bone- and tissue-compatible, and
selectively removing said pyrocarbon coating from regions of said body to expose said open cell reticulated structure and thereby promote bony and/or tissue ingrowth into such selected regions when said body is implanted in association therewith.

11. The method of claim 10 wherein said biomaterial is a lightweight substrate having a surface a thin film of metal throughout the network.

12. The method of claim 11 wherein said selective removal is carried out without removing the underlying metal covering so as to expose said substrate.

13. The method of claim 12 wherein said selective removal is by electrodischarge machining.

14. The method of claim 13 wherein said metal is tantalum.

15. The method of claim 10 wherein said deposition of pyrocarbon is carried out to deposit a layer having a thickness sufficient to allow it to be polished and serve as an articulating surface in a bone joint.

16. The method of claim 15 wherein said pyrocarbon layer has a thickness of at least about 0.2 mm.

17. The method of claim 16 wherein said pyrocarbon is pure unalloyed carbon which has a density between 1.7 and 2.1 grams per cm³, a K_{ic} of at least about 1.2 MPa (√ m), a modulus of rupture for bending of at least about 58 Kg/m² psi×10³, and a Diamond Pyramid Hardness of between about 200 and 250.

18. The method of claim 15 wherein said pyrocarbon is polished in a region to serve as an articulating surface having surface irregularities not greater than about 0.01 mm.
